(19) United States
(12) Patent Application Publication
(10) Pub. No.: US 2017/0296054 A1
(43) Pub. Date: Oct. 19, 2017

(54) WIRELESS MONITORING DEVICE

(71) Applicant: iMobile Healthcare, LLC, West Palm Beach, FL (US)

(72) Inventors: Aaron GOLDSTEIN, West Palm Beach, FL (US); Collin HILL, Greenwich, CT (US); William DUCKWORTH, Lake Forest, IL (US)

(21) Appl. No.: 15/629,609

(22) Filed: Jun. 21, 2017

Related U.S. Application Data
(63) Continuation-in-part of application No. 14/282,469, filed on May 20, 2014.
(60) Provisional application No. 61/825,173, filed on May 20, 2013.

(51) Int. Cl. A61B 5/00; A61B 5/01

(52) U.S. Cl. CPC A61B 5/0008; A61B 5/746; A61B 5/01; A61B 5/4812; A61B 5/6833; A61B 5/742; A61B 5/0022; A61B 2560/0209; A61B 2560/0214; A61B 2560/045; A61B 2562/0219; A61B 2562/0271

(57) ABSTRACT

The present disclosure relates to a monitor and monitoring system suitable for attachment to the skin of a mammal, including a human. The monitor and monitoring system are designed for continuous wireless real-time measurement of physiological signals and transmission of the measurements through the cloud to a remote computer or mobile device.

### CROSS REFERENCE TO RELATED APPLICATIONS

This application claims priority to U.S. patent application Ser. No. 14/282,469 filed May 20, 2014 which claims benefit of U.S. Provisional Application No. 61/825,173 filed May 20, 2013, each of which is incorporated by reference herein in its entirety.

### FIELD OF THE INVENTION

A device and method for continuously monitoring an individual's physiological signals.

### BACKGROUND

Health monitoring allows for the discovery and treatment of ailments early in the progression of an illness and helps prevent treatable conditions from becoming life threatening. However, most monitoring is performed intermittently, and if an individual's condition changes rapidly, such changes may not be recognized in time for appropriate intervention to occur.

For example, chemotherapy patients have depressed immune systems and are at high risk for infection. A fever during chemotherapy treatment is considered a medical emergency (Division of Cancer Prevention and Control, National Center for Chronic Disease Prevention and Health Promotion, Jun. 25, 2013), however effective intervention requires that a patient realize they have a fever before it becomes life threatening. Children who are prone to febrile seizures may need continuous monitoring at home to allow for intervention prior to a seizure occurring. Fevers in elderly, frail, or debilitated individuals are frequently a sign of a severe infection that should be treated immediately (Keating M J III., Klimek J J, Levine D S, et al. Effect of aging on the clinical significance of fever in ambulatory adult patients. J Am Geriatr Soc 1984; 32:282-7). Additionally, vital sign monitoring may be important after surgery, even if a patient has been released from a hospital. For example, after surgery, such as surgery undertaken to restore blood flow to an injured body part, it may be necessary to continuously monitor the area below the injury for temperature and continued circulation.

Traditional thermometers include a liquid that expands or otherwise changes its physical conformation when heated. Thermoresistors in digital thermometers change resistance with changes in temperature which can then be measured and converted to a numerical reading. Aural thermometers use an infrared sensor to measure temperature. However, all of these are designed for intermittent monitoring and require disturbing the patient in order to obtain a reading. There is therefore a need for a means to wirelessly measure temperature and other physiological signals on a continuous basis without disturbing the patient.

### SUMMARY

Provided herein is a means for continuously monitoring one or more physiological signals in mammals including humans. As used herein, physiological signals, including vital signs, are clinical measurements that indicate the state of a patient's body functions. Such monitoring may be used for a variety of purposes, including for the detection, tracking, and diagnosis of particular diseases or conditions.

In some embodiments, provided herein is a means for continuously monitoring individuals at risk for rapid changes in physiological states and detecting the rapid changes in physiological states. While any individual may be monitored, in some embodiments, individuals being monitored include, but are not limited to, those at risk for seizures or suffering from acute infection. In some embodiments, the continuous monitoring system and methods may be used in sleep diagnostics, including in the diagnosis of sleep apnea.

Further provided herein is a means for self-contained non-invasive wireless continuous monitoring using a sensor assembly comprising one or more sensors encased in a sensor housing and a means for attaching the sensor housing to the body. The sensor housing and one or more sensors in the sensor housing are placed proximate to or against the skin surface of a mammal including a human. In some embodiments, one or more sensors in one or more sensor housings may be placed at one or more locations on the individual. In some embodiments, each sensor may have and/or may transmit a unique identification identifying the individual being monitored, the type of condition being monitored, or parameters within which sensors measurements should stay between, not fall below, or not exceed.

The sensor may be an infrared sensor, thermistor, pulse oximeter, EKG monitor, cardiac telemetry monitor, blood pressure monitor, heart rate monitor, respiration rate monitor, body temperature monitor, accelerometer and/or an electrocardiogram monitor. In some embodiments, the sensor may transmit a signal to a processor such as a CPU, microprocessor or microcontroller contained within the sensor housing. The sensor measurement is then wirelessly transmitted to a local relay unit using low energy transmission. The sensor may transmit the signal directly to the relay unit, or send it through other processes in the sensor housing unit. In some embodiments, the relay unit may track physiological signal trends, activate an alarm when pre-determined parameters are exceeded, display the vital signs or other physiological signals, display trends in physiological signals and/or transmit the sensor reading through the cloud to a remote second display device such as a computer, personal digital assistant, mobile device or tablet. The cloud is generally interpreted to mean an internet based computing system where different services, including, but not limited to, servers, storage and applications, are delivered to an organization's computers and devices through the Internet. In some embodiments, the relay unit transmits information to a server via the internet, an intranet, private or public networks and the like and the data is then further transferred through the cloud by the server to a remote second display device such as a computer, PDA, smart phone or tablet. The server may track vital sign trends or other physiological signals, activate an alarm when pre-determined parameters are exceeded, display the physiological signals, and/or further transmit the sensor reading to a remote second display device such as a computer, mobile device or tablet. In other embodiments, the relay unit transmits information directly to one or more remote second display devices through the cloud. In some embodiments, the remote second display device may have an application that allows the remote device to track vital sign or other physiological signal trends, activate an alarm when pre-determined parameters are exceeded, and/or display the physiological signals or trends in physiological signals. In additional embodiments, receipt of sensor readings from the relay unit by the remote second display device that exceed pre-set parameters may trigger an alarm on the remote device. In another embodiment, sensor readings that exceed pre-set parameters may trigger a message sent to emergency services including, public organizations that respond to and deal with emergencies when they occur, especially those that provide police, ambulance, and firefighting services. In some embodiments, a message may be sent to someone monitoring the individual under observation.

These and other embodiments, features and potential advantages will become apparent with reference to the following description.

### BRIEF DESCRIPTION OF THE DRAWINGS

FIG. 1 is a side view of an embodiment of a sensor housing unit with a sensor.

FIG. 2 is a view of a sensor housing unit and an adhesive patch.

FIG. 3 is a view of the inside of an embodiment of a sensor housing unit.

FIG. 4 is a view of an embodiment of a sensor holder.

FIG. 5 is a view of a system for transmitting physiological signals.

FIG. 6 is a view of an embodiment of a relay unit.

FIG. 7 is a view of a system for transmitting a physiological signal to a relay unit for display.

FIG. 8 is a view of an embodiment of a system for transmitting physiological signals.

### DETAILED DESCRIPTION

Provided herein is a means for continuous, non-invasive, real-time wireless monitoring of one or more of an individual's physiological signals in inpatient settings, outpatient settings or both. Further provided herein is a means for sending an alert to emergency services and/or a person monitoring the individual under observations when an individual's physiological signals or a trend in the individual's physiological signals exceed specific parameters. In some embodiments, a means for transmitting data to electronic medical records directly or through an intermediary device such as through the cloud to an existing hospital server which has an interface with the electronic medical records system is provided.

The system described herein may be used to continuously monitor one or more vital signs such as the four primary vital signs: body temperature, blood pressure, heart rate, and respiratory rate; as well as additional physiological signals such as movement, brain wave activity, or those characterized as the “fifth vital sign” or “sixth vital sign.”

The physiological signals are measured using a system comprising a reusable sensor housing, at least one, two, three, four, or more reusable physiological sensors, a user replaceable battery, wherein the battery may be rechargeable or non-rechargeable, a power button or other means for activating the device such as through an application located on the relay unit and/or remote second display device, a microcontroller, a power controller, a communication chip, a disposable means for attachment, and a relay unit which may display information locally and/or transmit information through the cloud to a remote second display device. The sensor in the sensor housing may measure physiological signals including, but not limited to, temperature, blood pressure, oxygen levels, electrical conduction, pulse, respiration, heart rate and rhythm, activity levels and the like which may be referred to herein as physiological signs. The sensor and the sensor housing are unable to display any physiological data without the use of another separate, additional device such as the relay unit. The sensor or sensor housing unit transmits data and communicates with the relay unit using low power, short range wireless communication protocols. The relay unit may display the data and/or transmit the data through the cloud to a remote second display device for access by an interested party. The relay unit acts as a first point of internet or wide area network connectivity for the sensor device. In some embodiments, the relay unit receives and transmits information to the sensor housing unit to control the functionality of the sensor unit. The relay unit comprises both low energy, short range wireless communication and a means for connecting to a cloud network. Signals may be sent from the relay unit to the cloud using any form of internet connectivity including, but not limited to, Wi-Fi, Ethernet or cellular connectivity of any kind. In some embodiments, the relay unit may display sensor information to the user. In displaying physiological measurements, the relay unit may use short range, low energy data transmission.

In some embodiments, if the sensor measurement exceeds pre-set parameters or a trend in the sensor measurements exceeds pre-set parameters, an alarm on either or both the relay unit and remote second display device may be triggered. In some embodiments, if the sensor measurement exceeds pre-set parameters, emergency services may be notified. For example, in some conditions, such as seizures, there are monitorable symptoms that may appear just prior to onset of the seizure such as daydreaming episodes in which activity in certain areas of the brain increase, jerking movements of the arm, leg or body, unexplained sleepiness or weakness, and falling. Additionally, in some instances, seizures may be preceded by a rapid change in temperature, either an increase or decrease, directly prior to the onset of seizure. An alarm triggered by indications of one or more of these symptoms in an individual at risk for seizures may provide enough time for the individual to stop what they are doing or get to a safe location for the approaching seizure or enough time for an intervention to prevent a seizure.

In the instance of infections, individuals may be monitored for the appearance of an infection through the use of one or more physiological sensors. For example, in some infections such as sepsis, a physiologic response includes an abnormally high temperature or low temperature and one or more of rapid heart rate and rapid breathing rate, and decreased blood pressure. Changes in the state of an individual with sepsis require an immediate response. There are also individuals who may be at risk for sudden, acute deterioration in their condition or rapid changes in temperature who may be monitored using the sensor unit described herein. Such monitoring may take place in inpatient or outpatient settings.

For example, in inpatient settings, the system allows for one or more patients to be monitored simultaneously. In some embodiments, a single patient is monitored. In other embodiments, a plurality of patients may be monitored, significantly reducing the cost of monitoring. Multiple wireless sensor units may be placed on one or more patients. A relay unit is placed in each room. The relay unit forwards data received from the sensor assembly to a cloud server system. The data forwarded from the relay unit may be displayed on the relay unit and/or one or more remote second display devices. In some embodiments, the relay unit may transmit data through the cloud to interface with existing electronic medical record data. While, this embodiment is described for use in inpatient settings, it may also be deployed in outpatient settings such as situations of natural disasters or other areas with multiple individuals under care and low resource availability.

In outpatient settings, the system may be used to monitor a single individual with one or more sensor assemblies attached to the body and a relay unit in the room with the individual. The relay unit transmits information received from the sensor assembly through the cloud to a second display device.

In other embodiments, the system described herein comprising a sensor unit, relay unit, cloud, and remote second display device may be used in telemedicine for remote diagnosis and treatment by means of telecommunications technology. The sensors in the sensor unit supply real-time data to the relay unit. The relay unit transmits the data through the cloud to the remote second display device. The caregiver such as a doctor, nurse, or nurse practitioner, uses the real-time data supplied by the sensors in diagnosing and/or preparing a treatment plan for a condition in an individual.

In additional embodiments, the sensor unit described herein may be used to monitor sleep quality such as the amount of time spent in a sleep cycle such as REM sleep or the amount of movement an individual experiences during sleep. As you enter a period of REM sleep, thermoregulatory efficiency decreases. Therefore, if you are in a cold environment your body temperature will drop dramatically and vice versa in a warm environment. By measuring changes in body temperature over time, the overall quality of sleep can be determined. Additionally, an accelerometer may be used to detect body positioning and movement which provides additional data to a sleep study, such as in the diagnosis of sleep apnea.

The sensor unit may be attached to the body by any means generally used including disposable adhesive patches or straps. In some embodiments, multiple wireless sensor units may be placed at different points on the body with the same or different sensors and the information may be aggregated to provide a more complete picture of an individual's condition. The sensor may have or may transmit a unique identification so that information from different sensors on an individual or different sensors on multiple individuals may be distinguished. The unique identification may be any type of identification generally used including, but not limited to, patient identification numbers, sensor numbers such as 1, 2, 3, or specific bar code type sensor information for each sensor, or any combination thereof. For example the unique identification may be a code identifying the individual being monitored and the placement and/or identification number of the sensor.

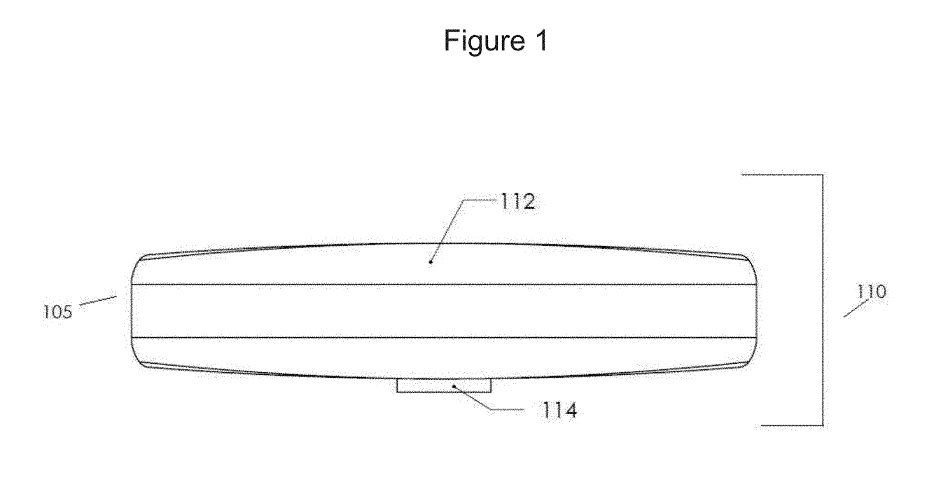

As shown in FIG. 1, a sensor unit 110 comprises a sensor housing 105, a power source and one or more wireless reusable physiological sensors 114. In some embodiments, the physiological sensor 114 may be flush with the sensor housing 105. In other embodiments, the physiological sensor 114 may protrude or extend from a first side 116 of the sensor housing 105 as shown in FIG. 1. In additional embodiments, the sensor unit 110 may be waterproof. In other embodiments, the sensor may be housed completely within the sensor unit. For example, if the sensor unit contains a plurality of sensors such as a thermistor and accelerometer, the thermistor may extend or protrude from the sensor housing to permit it to be in direct contact with the skin, while the accelerometer may be completely housed within the sensor unit.

The term “sensor” as used herein refers to any component that is capable of detecting physiological changes through the skin of an individual. Sensors may include any type of electrical, optical, mechanical, and/or chemical non-invasive sensors. The physiological sign sensor 114 may be any type of sensor useful in continuous monitoring, including, but not limited to, an infrared sensor, thermistor, pulse oximeter, accelerometer, EKG monitor, cardiac telemetry monitor, blood pressure monitor, heart rate monitor, respiration rate monitor, body temperature monitor, electrocardiogram monitor and the like.

In some embodiments the sensor housing 105 is round as shown in FIG. 1. In other embodiments, the sensor housing is rectangular. In further embodiments the sensor housing is oval. In additional embodiments, the sensor housing is any regular or irregular geometric shape.

The sensor unit may be powered by any means generally used. In some embodiments, the sensor may be powered by an externally accessible replaceable battery 112 located on a second surface of the sensor housing 105 of the sensor unit 110, or within the sensor housing 105, or accessible from the second surface of the sensor housing 105. The battery may be user-replaceable. In some embodiments the battery may be a standard battery. In other embodiments, the battery may be rechargeable. In further embodiments, the sensor may be powered by inductive coupling. In additional embodiments, the sensor may be rechargeable such as through the use of a USB port which plugs into the sensor unit 110, a charging plate, or similar devices.

The sensor housing 105 may additionally encase a replaceable battery, a microcontroller, a communication chip, and a power controller. A wireless continuous monitoring sensor assembly comprises the sensor housing and its components along with a means for attaching the sensor housing to an individual, such as to the core of an individual. In some embodiments, the sensor may be on a first side of the sensor housing and the battery is on or accessible from a second side of the sensor housing. In some embodiments, at least one sensor may be located completely or partially within the sensor housing. The sensor data is transmitted to a relay unit which then sends the data through the cloud to a remote second display device.

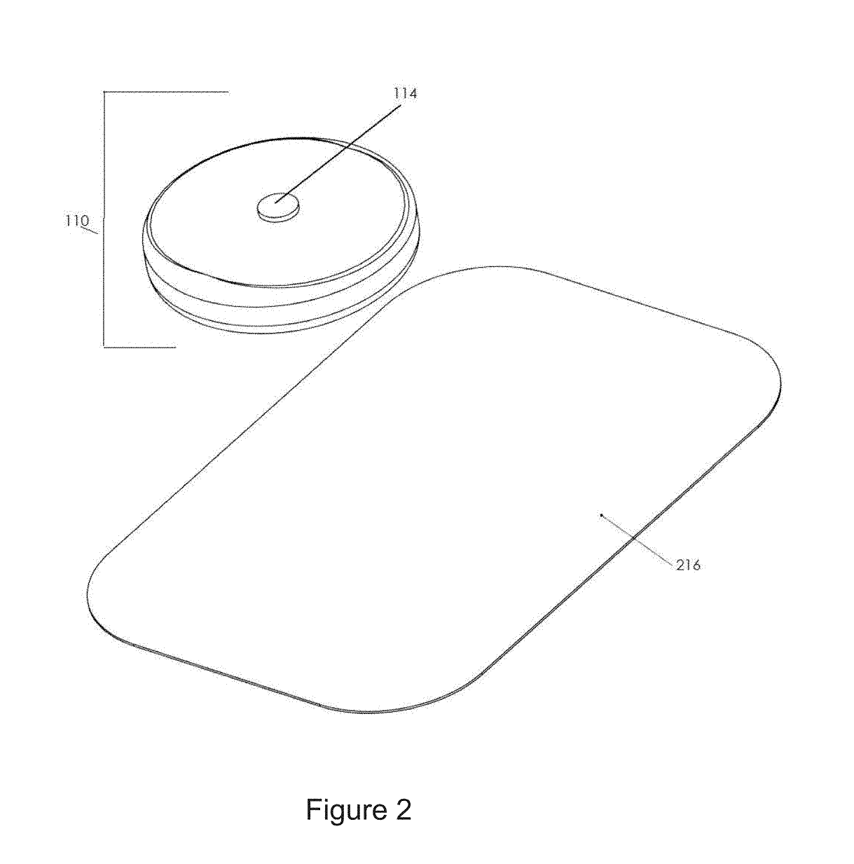

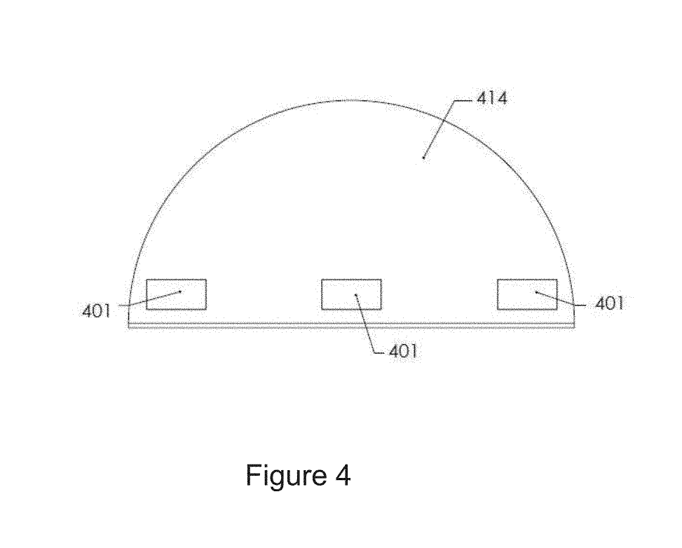

The physiological sensors described herein are designed to be worn continuously by the individual in need of monitoring. The physiological sensors may be attached to an individual by any means generally used. In some embodiments, the physiological sensor may be an epidermal electronic. In other embodiments, the sensor unit is part of a sensor assembly which includes a means for selectively mounting the sensor unit on the skin. In some embodiments, a sensor unit 110 with a physiological sensor 114 is attached using an adhesive patch 216, such as the one shown in FIG. 2, to form a sensor assembly. In further embodiments, the sensor unit may be worn beneath an item of clothing. In another embodiment, the sensor and/or the sensor unit may be incorporated into wearable jewelry such as a wrist band or chest band. In yet another embodiment, the sensor and/or sensor unit may be incorporated into an item of clothing such as a sock, shirt, pajamas, hat, onesie, or a glove. In some embodiments it may be incorporated into a sensor holder such as the skull cap as shown in FIG. 4. In some embodiments the means for attaching the sensor housing to the body is disposable. In other embodiments, the means for attaching the sensor housing to the body is reusable.

In some embodiments, the sensor unit 110 may be a one-time-use sensor unit that is provided in a sealed sterile package. In other embodiments, elements of the sensor unit 110 can be disposable while some components are reusable. For example, in some embodiments, the sensor unit 110 may have an externally accessible replaceable battery 112. In other embodiments, the sensor unit 110 may be rechargeable, for example through a USB port. In a further example, the physiological sensor 114 may be replaceable. In additional embodiments, the physiological sensor 114 may be reusable. In some embodiments the sensor unit may have an on/off switch. In other embodiments, the sensor unit may automatically turn on when placed in contact with the skin. In additional embodiments, the sensor may be disposable while the sensor housing is not. In further embodiments, the only portions of the sensor unit that require replacement are the batteries and/or the means of attachment.

The sensor unit may be attached anywhere on the body that is useful in measuring physiological signs. In some embodiments, the sensor unit 110 may be selectively attached to the patient's forehead, armpit, arm, chest, foot, abdomen, hand, or back of the ear. In other embodiments, the sensor unit 110 is selectively attached to the body's core (i.e. the body without its arms and legs). In some embodiments, the sensor unit 110 is placed so that the physiological sensor 114 has continuous contact with the skin. In other embodiments, the sensor housing 105 is placed so that the physiological sensor 114 is proximate to the surface of the skin. In some embodiments, multiple wireless sensor units are attached at multiple locations on the same or different individuals. In additional embodiments, each sensor may have or transmit a unique identification code. In some embodiments, skin surface measurements may be taken. In other embodiments, core temperature measurements may be taken.

In some embodiments, the sensor unit 110 can include an adhesive backing that helps to facilitate and maintain placement of the sensor by removeably adhering to the patient's skin. In another embodiment, the sensor can comprise adhesive backed foam. The adhesive backing can also help to maintain sensor contact with the user's skin for those sensors that require skin contact. According to some embodiments, conductive sensors may have a conductive gel placed over these sensors.

In some embodiments, the sensor unit may be adhered to the skin using a disposable adhesive patch. The patch may be designed with an adhesive to stay affixed to the skin for 1 or more days, up to 2, 3, 4, or more days. While the patch may be any size, generally the patch is as small as possible, yet still provides enough adhesion to hold the sensor in place. In some embodiments the area of the patch is less than about 1 square inch. In other embodiments, the patch may be about 1 inch in diameter. The patch may be circular, oblong or any other regular or irregular geometrical shape. In some embodiments, the patch may be colorful and have designs or cartoon pictures. In some embodiments, the sensor unit many about 5 inches by about 5 inches, 3 to about 3 inches or smaller, for example, it may be 1 by 2 inches or smaller, 0.75 to 1.5 inches or smaller, 1 inch by inches or smaller, 0.025 by about 0.25 inches or smaller, or any subset thereof. The adhesive size of sufficient size to hold the sensor unit securely in place. For example, in some embodiments, the patch may be about 3 to 4 inches in size to hold a sensor unit of 1 by 2 inches, though larger patches may also be used depending on the requirements of securing the device.

In some embodiments, the sensors may be part of a patch. In other embodiments, the patch may be placed over the sensor housing as part of a sensor assembly. The patch may be designed with an adhesive to stay affixed to the skin for 1 or more days, up to 2, 3, 4, or more days. While the patch may be any size, generally the patch is as small as possible yet still provides enough adhesion to hold the sensor in place. In some embodiments the area of the patch is less than about 1 square inch. In other embodiments, the patch may be about 1 inch in diameter. The patch may be circular, oblong or any other regular geometrical shape or irregular shape. In some embodiments, the patch may be colorful and have designs or cartoon pictures.

The combination of the patch and sensor have a thickness, ranging from 0.5 mm to about 8 mm, from about 5 mm to 7 mm, and about 6.4 mm. The patch may include a body composed of a polymeric material such as a neoprene rubber. In other embodiments, the sensor is part of an epidermal electronic with a thickness of about 1 to about 4 μm.

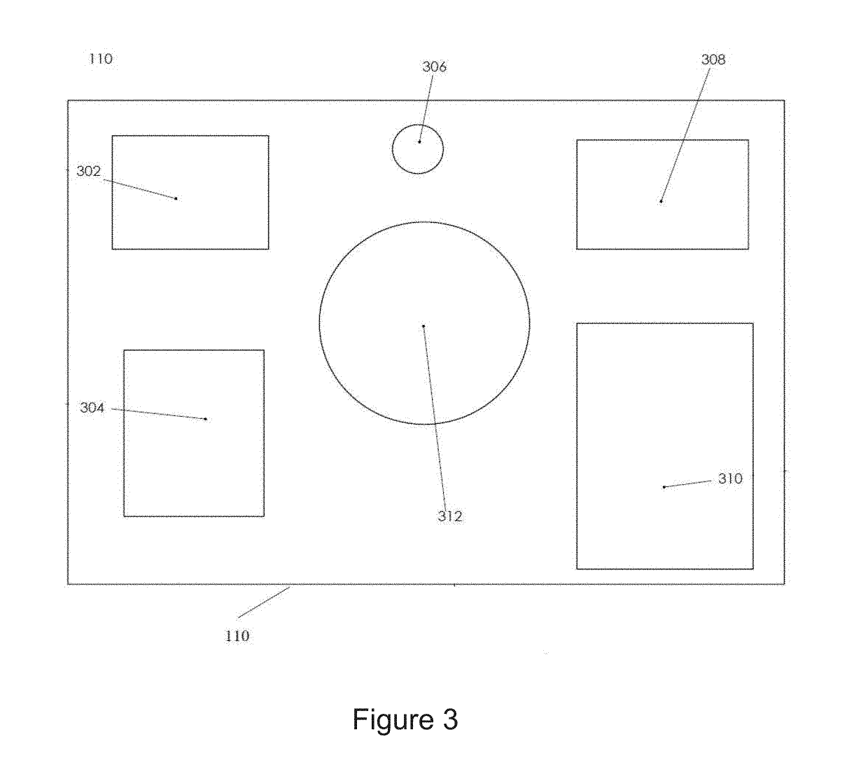

As shown in FIG. 3, the sensor unit 110 may comprise a memory unit 302, an antenna 304, one or more physiological sensors 306, power controller 308, and a CPU 310. In some embodiments, the physiological sensor 306 may be a thermistor. A thermistor is a temperature-sensing element composed of sintered semiconductor material which exhibits a large change in resistance proportional to a small change in temperature. In some embodiments the thermistor measures core temperature. In other embodiments, the thermistor measures skin temperature. In additional embodiments, the sensor is an infrared sensor, pulse oximeter, EKG monitor, cardiac telemetry monitor, accelerometer, blood pressure monitor, heart rate monitor, respiration rate monitor and the like. In some embodiments, the sensor unit 110 may comprise multiple sensors including 1, 2, 3, 4, 5, or more sensors. Each of the multiple sensors may be the same or different depending on what needs to be monitored in the patient and may protrude from or be entirely encased by the sensor housing. In some embodiments, a patient may wear more than one sensor unit in more than one place on the body. The sensors in each sensor unit may be the same or different depending on the needs of the patient.

The antenna 304 may transmit a signal by any means generally used. In some embodiments, the antenna 304 may use a wireless protocol. For example, the antenna may use sub-GHZ, ZigBee, Bluetooth, passive RF, or Wi-Fi. In other embodiments, a signal may be sent using infrared or ultrasound wireless control. The antenna length needed for operating at different frequencies is 17.3 cm at 433 MHz, 8.2 cm at 915 MHz, and 3 cm at 2.4 GHz. The 2.4 GHz band has the advantage of enabling one device to serve in all major markets worldwide since the 2.4 GHz band is a global spectrum. However, 433 MHz is a viable alternative to 2.4 GHz for most of the world, and designs based on 868 and 915 MHz radios can serve the U.S. and European markets with a single product. In some embodiments, the frequency may be 14.46 MHz. The antenna may be straight, coiled, or in any configuration useful for transmitting a signal. In some embodiments, the antenna 304 may be replaced with a transceiver. In other embodiments, the physiological sensor signal may be amplified before being transmitted.

The memory unit 302 may be used to store raw measured or processed physiological signals. In some embodiments, the memory unit 302 may store trends in changes in the patient's physiological signs. In other embodiments, the memory unit 302 may compare changes in temperature readings to determine the rate at which an individual's temperature is increasing. In additional embodiments, the memory unit 302 may store data until a physiological sign exceeds certain parameters at which point the sensor unit sends a signal to a relay unit which may analyze the information or transfer the information directly to a computer or mobile device for analysis.

In some embodiments, the CPU 310 takes in raw voltage or resistance data from the thermistor, converts it into useable temperature data and then provides new binary temperature data to be transmitted to the relay unit.

In some embodiments, the memory unit 302 and the CPU 310 may be replaced by a microcontroller. The microcontroller may include a CPU storage/memory (e.g., RAM, ROM, EEPROM, flash), general purpose input/output (GPIO), analog-to-digital (A/D) and digital-to-analog (D/A) converters, as well as digital signal processors (DSP).

In some embodiments, the sensor unit may further comprise a communication chip that transmits data processed by the signal from one or more physiological sensors and transmits the data to the relay unit.

In some embodiments, the power controller 308 may be an intelligent selection of transmitter power output in a communication system to achieve good performance within the system. In other embodiments, the power controller 308 may be a proportional-derivative-integrative controller.

The power source 312 may be a permanent, replaceable or rechargeable battery. In some embodiments, the sensor unit 110 may be recharged using a USB port or other similar device. In additional embodiments, the power source 312 may be rechargeable using a charging plate. In additional embodiments, the power source may be replaced by the patient as needed.

FIG. 4 depicts an additional embodiment of a sensor unit. As shown in FIG. 4, a skull cap 414 houses one or more vital sign or physiological sensors 401. The sensors may be any type of vital sign or other physiological sensor useful in continuous monitoring, including, but not limited to, an infrared sensor, thermistor, accelerometer, pulse oximeter, EKG monitor, cardiac telemetry monitor, blood pressure monitor, heart rate monitor, respiration rate monitor, and the like. The physiological sensors 401 may be the same or different. In some embodiments, the physiological sensors 401 are equally spaced on the skull cap 414 as shown. In other embodiments, a single physiological sensor 401 is placed in the skull cap 414. In additional embodiments, a plurality of physiological sensors 401 may be placed as needed throughout the skull cap.

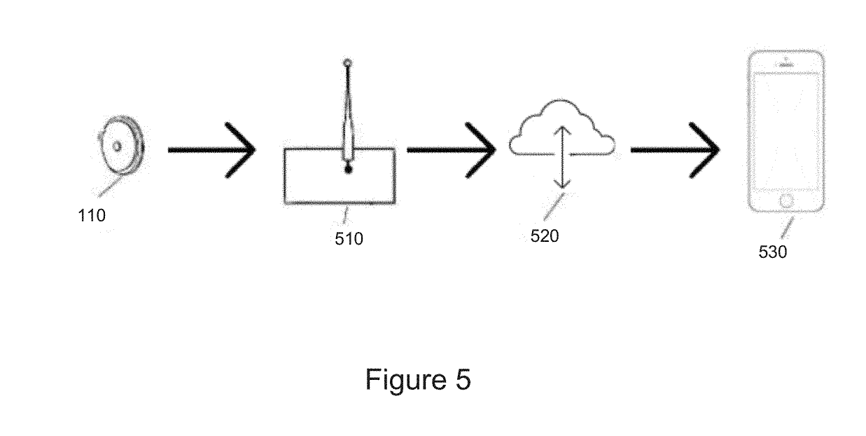

As shown in FIG. 5, the readings from the sensor unit 110 are sent to a nearby relay unit 510. Readings may be sent from the sensor unit 110 by any means generally used to transmit low power signals over short distances including, but not limited to, sub-GHZ, ZigBee, Bluetooth, passive RF, or Wi-Fi. In other embodiments, a signal may be sent using infrared or ultrasound wireless control. In some embodiments, the relay unit 510 may record multiple physiological sensor readings. In other embodiments, the relay unit 510 may determine trends based on physiological sensor readings. In some embodiments, the relay unit 510 may display the physiological sensor measurements on a first surface of the relay unit so that the relay unit is functioning as a first display device as well as receiving the low energy sensor transmissions. In other embodiments, the relay unit 510 merely transmits the information to a remote second display device 530 such as a computer, tablet, and mobile device, or similar devices via the cloud 520. In some embodiments, the relay unit 510 may send a signal to a remote server through the cloud 520. In some embodiments, the remote server may be part of a private network. In another embodiment, the signal may be sent via the internet. In further embodiments, it may be sent over a secured line. In additional embodiments, the signal may be encrypted. The relevant information from the sensor unit 110 is then sent from the server to the remote device. The information may be displayed by any means generally used. In some embodiments, the information is sent via a text message. In another embodiment, the information is sent by the remote server to an application on the remote second display device. In a further embodiment, the information is sent via a pre-recorded message from the remote server. The remote server may send as little or as much information as desired. In some embodiments, the server only sends information to the remote second display device when certain parameters are exceeded. In other embodiments, the server may continually update the remote second display device. In additional embodiments, the remote second display device may be periodically updated. In some embodiments, the information from the sensor may trigger an alarm in the relay unit and/or remote second display device when the data exceeds certain parameters. For example, if temperature is being measured, an alarm may trigger if the sensor measures a temperature below 95° F. or above about 100° F. In the case of a child, an alarm may be triggered if the temperature of the child reaches 102° F. In another embodiment, an alarm may be triggered if particular trends are noted even if the sensor is not measuring a temperature exceeding pre-set parameters. For example, if an individual's temperature is trending upwards over a certain period of time, an alarm may be triggered. In additional embodiments, a processor in the relay unit and/or in an application in the remote device may compensate for normal fluctuations in vital and other physiological signs. For example, body temperature normally fluctuates by almost a degree Fahrenheit during the course of the day with the body temperature lower in the morning and higher in the evening. Therefore, a slight upwards trend in temperature during the course of the day may not trigger any sort of alarm in the remote second display device. In other embodiments, an alarm may be triggered if a particular trend is observed. In some embodiments, if the sensor reaches a particular threshold, emergency services or a doctor's office may be contacted. Parameters for an alarm to be triggered may be pre-set by the manufacturer or may be set by the individual monitoring the sensor wearer. In some embodiments, the relay unit, an optional remote server, and/or remote second display device may store historical data from the sensor(s) allowing production of the sensor data for a physician. In some embodiments, information from the sensors may be available through a web portal.

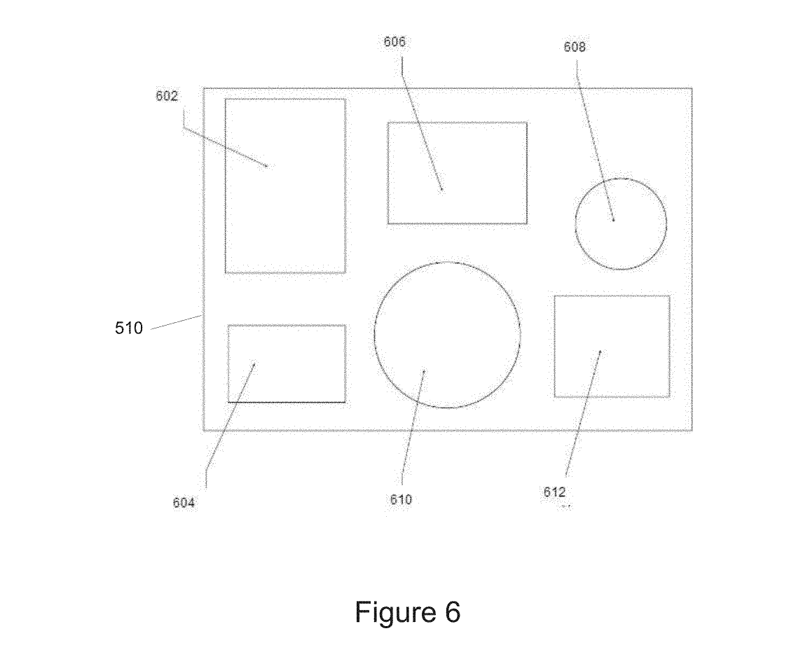

As shown in FIG. 6, the relay unit 510 may comprise a CPU 604, a transceiver 612, a power controller 602, and a Wi-Fi radio 606. In some embodiments, the relay unit 510 may plug into a wall to obtain power. In other embodiments it may use a replaceable battery 610. In further embodiments it may be rechargeable, for example through a USB port.

The CPU 604 is the component controlling other components in the relay unit 510. In some embodiments, it may analyze the data from the sensor. For example, in some embodiments, the physiological sensor readings may be converted to either analog or digital numbers for display by the CPU. In other embodiments, the conversions make take place in the relay unit. In a further embodiment, the conversion may take place in the remote second display device. In general, the more speed and data analysis required, the more power is needed. Therefore a sleep function is often used in order to save power. At certain times or if certain events happen, the CPU wakes up, makes the necessary calculations, communicates with relevant components and returns to sleep mode. In some embodiments, the activity of the CPU may be controlled by a signal sent by the relay unit.

The power controller 602 selects transmitter power output to achieve good performance within the communication system. The transceiver 612 may be any type of transceiver generally used. In some embodiments it may comprise a radio with an antenna. The Wi-Fi radio 606 takes the signal received and sends it to a remote server.

The relay unit 510 may additionally comprise a status light 608. Such a status light may change colors when charging, when turned on, when a signal is being sent, when a signal is being transmitted, or any additional status desired. The status light may convey information by remaining steady, blinking, blinking in a particular pattern, displaying a particular color, turning off or any other means status lights convey information.

In some embodiments, the relay unit 510 may additionally store information received from the sensor. In other embodiments, the relay unit 510 may analyze the information received from the sensor and compare it to previous readings to determine if there is a trend in the physiological sign, particularly a trend indicating there is an issue with the individual being monitored.

In some embodiments, the sensor unit 110 may be remotely activated. For example, it may be less necessary for physiological signs to be monitored while a patient is awake or if the patient is hospitalized. The sensor may therefore be programmed by the relay unit 510 or the remote second display device 530 to turn on or off at certain times of day. In other embodiments, the sensor unit 110 may be switched on or off remotely and/or manually.

In some embodiments, the physiological signs of an individual are monitored continuously during real time by attaching a sensor in a sensor unit to an individual, measuring the physiological signs and sending the measurements to a relay unit. The data from a physiological signal may be sent to the relay unit using sub-GHZ, ZigBee, Bluetooth, passive RF, or Wi-Fi. In other embodiments, a signal may be sent using infrared or ultrasound wireless control. In some embodiments, the relay unit may record and analyze the information received from the sensor. The information is then sent from the relay unit to a remote second display device through the cloud which may sound an alarm if a pre-determined parameter is exceeded. In other embodiments, the relay unit and/or remote second display device may contact emergency services if the pre-determined parameter is exceeded.

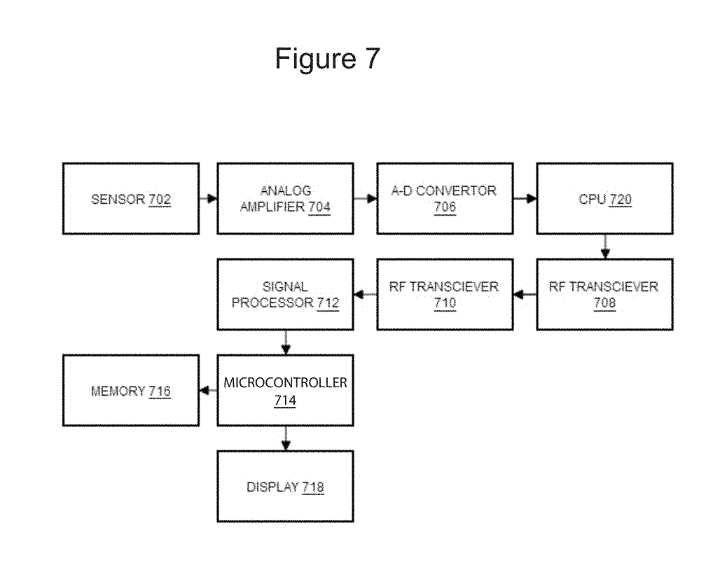

As shown in FIG. 7, in some embodiments a physiological signal is measured by a sensor 702. The signal from the sensor 702 is amplified by an analog amplifier 704. The analog signal from the analog amplifier 704 is converted to a digital signal by an A-D convertor 706. The digital signal is then sent to a CPU 720 for processing. After processing, the signal is sent to a first RF transceiver 708 and then to a second transceiver 710 in a relay unit. The signal from the second RF Transceiver 710 is then processed by a signal processor 712 to optimize it and the resulting physiological measurement is analyzed by the microcontroller 714, stored in the memory 716 and displayed 718 on a first surface of the relay unit.

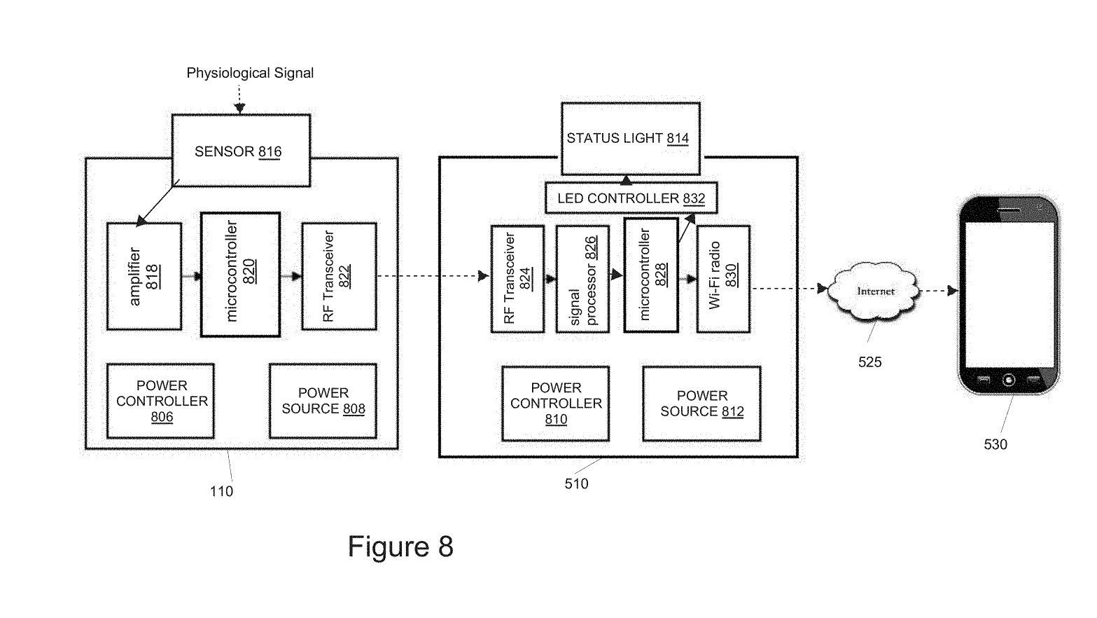

In other embodiments, as shown in FIG. 8, a sensor 816 in a sensor unit 110 measures a physiological signal. The sensor 816 sends the signal to an amplifier 818. The amplifier 818 transmits the signal to the microcontroller 820. The microcontroller 820 converts the signal from analog to a digital signal, processes the signal, records the signal and transmits it to the RF transceiver 822. The RF transceiver 822 sends the signal to a second RF transceiver 824 in the relay unit 510. The RF transceiver 824 then sends the signal to a signal processor 826 which improves the accuracy and reliability of the signal. The cleaned signal is then sent to a microcontroller 828 where the digital signal is converted to a physiological measurement and recorded. The sensor unit is powered by power source 808 which in some embodiments may be a user replaceable battery. In other embodiments, the power source may be a conventional battery. In some embodiments, the battery may be selected based on battery life. For example, in some embodiments, the battery may remain charged for minutes, days, hours, months or years. In some embodiments, unit power controller 806 allows the sensor unit to be activated manually. In some embodiments, unit power controller 806 may allow for remote activation of the sensor unit.

In some embodiments, the relay unit 510 may include a status light 814, generally an LED light. The microcontroller 828 sends a signal to the LED controller 832 to alter the status light as appropriate. Such a status light may change colors when charging, when turned on, when a signal is being sent, when a signal is being transmitted, or any additional status desired. The status light may convey information by remaining steady, blinking, blinking in a particular pattern, displaying a particular color, turning off or any other means status lights convey information. In other embodiments, data regarding the physiological measurements may be displayed. Power for the relay unit 510 is managed through power controller 810. In some embodiments, power controller 810 allows the relay unit to be activated manually. In other embodiments, relay unit power controller 810 may allow for remote activation of the relay unit. The physiological measurement is then sent from the relay unit to Wi-Fi radio 830 or transceiver which transmits the measurement to a remote server 525 through the cloud via Wi-Fi or other similar communication method. For example, the relay unit may send a signal to a remote server or remote second display device using Wi-Fi, SMS, WLAN, or a similar communication protocol. In some embodiments, the server may be part of a private network. In another embodiment, the signal may be sent via the internet. In further embodiments, the signal may be sent over a secured line. In additional embodiments, the signal may be encrypted. The remote server then sends the physiological measurement and/or any trends in measurements and other desired data to a remote second display device 530 such as a computer, tablet, mobile device, or similar device.

The relevant information from the sensor is then displayed on the screen of the remote second display device with or without trend analysis or other relevant analytics. In some embodiments, the information from the sensor may trigger an alarm in the remote device when the data exceeds certain parameters. For example, if temperature is being measured, an alarm may trigger if the sensor measures a temperature below 95° F. or above about 100° F. In the case of a child, an alarm may be triggered if the temperature of the child reaches 102° F. For example, if an individual's temperature is trending upwards over a certain period of time, an alarm may be triggered earlier. In additional embodiments, a processor in the relay unit and/or in an application in the remote device may compensate for normal fluctuations in physiological signs. For example, body temperature normally fluctuates by almost a degree Fahrenheit during the course of the day with the body temperature lower in the morning and higher in the evening. Therefore, a slight upwards trend in temperature during the course of the day may not trigger any sort of alarm in the remote device. In other embodiments, an alarm may be triggered if a particular trend is observed. In some embodiments, if the sensor reaches a particular threshold, emergency services or other parties may be contacted.

Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. “Hardware” refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations may involve optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microcontrollers), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory.

In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes and/or devices described herein, or a microcontroller configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

1. A system for wirelessly measuring and displaying the measurements of physiological signs in an individual comprising:
a self-contained non-invasive wireless sensor unit comprising:
at least one reusable physiological sensor extends from the self-contained non-invasive wireless sensor unit, wherein the at least one reusable physiological sensor measures a physiological signal from the individual;
a microcontroller, wherein the microcontroller processes the physiological signal from the at least one reusable physiological sensor;
a power controller;
a communication chip, wherein the communication chip transmits data processed by a signal from the at least one reusable physiological sensor and transmits data to a relay unit separate from the sensor unit;
a user replaceable battery;
a remote second display device to display the data, wherein the relay unit transmits the measurements from the at least one reusable physiological sensor through a cloud to the remote second display device; and
wherein the sensor unit is releasably attached to the individual.

2. The system of claim 1, wherein the at least one reusable physiological sensor is a thermistor.

3. The system of claim 2, further comprising a second physiological sensor, wherein the second physiological sensor is an accelerometer.

4. The system of claim 2, wherein the thermistor measures a core temperature.

5. The system of claim 2, wherein the thermistor measures a skin temperature.

6. The system of claim 1, wherein the relay unit receives information from the sensor unit through a low power transmission.

7. The system of claim 1, wherein if the sensor measurement of the physiological signal exceeds pre-set parameters, an alarm is triggered on the relay unit.

8. The system of claim 1, wherein if the relay unit detects a rapid change in the sensor measurement of the physiological signal, an alarm is triggered on the remote second display device.

9. The system of claim 1, wherein the system comprises multiple wireless sensor units on an individual, wherein each of the multiple wireless sensor units comprises at least two physiological sensors.

10. The system of claim 1, wherein the relay unit displays the sensor measurement on a first surface.

11. The system of claim 1, wherein the physiological sensor transmits a unique identification.

12. The system of claim 1, wherein if the sensor measurement of the physiological signal exceeds pre-set parameters, emergency services are notified by the remote second display device or the relay unit.

13. A wireless continuous monitoring sensor assembly comprising:
a sensor housing;
a reusable thermistor;
a reusable accelerometer;
a user replaceable battery;
a power controller;
a communication chip;
a disposable adhesive patch;
a microcontroller, wherein a CPU on the microcontroller processes data from the reusable thermistor and reusable accelerometer;
an adhesive patch;
a transmitter, wherein the transmitter sends data from the reusable thermistor to the relay unit; and
wherein the thermistor extends from a first side of the sensor housing such that the thermistor will be in non-invasive contact with a core of a body of an individual while the user replaceable battery, accelerometer, microcontroller and the power controller are maintained within the sensor housing; and
wherein the user replaceable battery is accessible from a second side of the sensor housing such that it does not interfere with a positioning of the thermistor.

14. The wireless continuous monitoring sensor assembly of claim 13, wherein the sensor housing is attached to the core of the body with an adhesive patch.

15. The wireless continuous monitoring sensor assembly of claim 13, wherein the relay unit comprises a means for sending the data from the thermistor through a cloud to a remote server.

16. The wireless continuous monitoring sensor assembly of claim 15, wherein the remote server transmits data from the thermistor and the accelerometer to a remote second display device.

17. A system for detection of a change in a physiological state in an individual comprising:
a self-contained non-invasive wireless sensor unit comprising:
at least two physiological sensors , wherein at least one of the physiological sensors extends from the sensor unit, wherein the at least two physiological sensors physiological sensor measure at least two different physiological signals;
a microcontroller;
a power controller;
a communication chip;
a user replaceable battery;
a cloud;
a relay unit, wherein the relay unit is separate from the sensor unit and wherein the relay unit transmits sensor measurements to the cloud; and
a remote second display device to display data, wherein the remote second display device receives data from the relay unit through the cloud and displays a change in a physiological state of the individual under observation.

18. The system of claim 17, wherein the individual has an infection.

19. A method of determining sleep quality comprising:
attaching at least one non-invasive sensor extending from a sensor housing to a body core of a mammal, wherein the sensor housing further comprises a microcontroller, a power controller, a communication chip, and a user replaceable battery;
measuring a change in thermoregulatory efficiency through a detection of a change in physiological signs of the mammal by the at least one non-invasive sensor in the sensor unit;
wirelessly transmitting the vital signs to a relay unit;
recording the physiological signs in the relay unit;
transmitting the vital signs from the relay unit through a cloud to a remote second display device;
wherein the remote second display device tracks physiological sign trends; and
wherein changes in thermoregulatory efficiency are indicative of different states of a sleep cycle.

20. The method of claim 19, wherein a decrease in body temperature in a cold environment indicates a period of REM sleep.
